(19) United States
(12) Patent Application Publication
(10) Pub. No.: US 2015/0191777 A1
(43) Pub. Date: Jul. 9, 2015

(54) RAPID IDENTIFICATION OF INFECTIOUS MICROORGANISMS AT POINT OF CARE

(71) Applicant: Adam D. Kennedy, Durham, NC (US)

(72) Inventor: Adam D. Kennedy, Durham, NC (US)

(21) Appl. No.: 14/151,009

(22) Filed: Jan. 9, 2014

(51) Int. Cl. C12Q 1/68; G06F 19/22

(52) U.S. Cl. CPC C12Q 1/689; G06F 19/22

(57) ABSTRACT

The present invention relates to the rapid identification of the species and subspecies in a sample such as a biological sample. By collecting and using PCR primers for the collection of species and subspecies and using PCR technology, the subspecies can be identified in 2-3 hours.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the identification of infectious disease. In particular it relates to the identification of a bacterial infectious disease clones and subclones in a rapid manner.

2. Description of Related Art

The methods of identifying a bacterial infectious disease are critical in the care of the medical patient as the distinctions of an infection as a part of acute care facilitates triage as well as treatment of the patient. Traditional hospitals and other locations based diagnostics identification methods include traditional culturing, high level PCR utilizing isolated DNA and antigen based diagnosis. These methods can diagnose to some degree but they have severe limitations, especially the time to complete the diagnosis. With a variety of clones with mutations existing and a variety of subclones rapidly being identified, the diagnosis of the exact infections agent can, in many cases, take 3 days or more. Since there are subclones that are deadly in a very short amount of time (hours rather than days) and there are antibiotic resistant subclones where other subclones are not, a quick diagnosis is critical but currently not available.

As one example, there are two major types of Staphylococcus aureus: community associated MRSA (methicillin-resistant S. aureus, CA-MSRA) and hospital associated MRSA (HA-MRSA). Detecting S. aureus by the MEC element (the genomic element that determines methicillin resistance) is not sufficient to discriminate between CA-MRSA and HA-MRSA. Additionally, there is a third type of S. aureus: live-stock associated MRSA (LA-MRSA), which has been transmitted from animals, particularly livestock, to humans. CA-MRSA strains are more virulent than HA-MRSA strains and infection with CA-MRSA strains can cause death within 24 hours. HA-MRSA strains are less virulent but are resistant to more antibiotics given the environment (hospital stays). Accordingly, there are adaptations of these subclones that allow each of them to cause infection and evade the immune response.

There is a need of a method for rapid diagnosis of the particular subclones of infectious bacterial strains so that treatment can be appropriately applied.

### BRIEF SUMMARY OF THE INVENTION

The present invention relates to the discovery that an unknown infectious bacteria present in an organism (e.g. mammal, human, etc.) can be identified down to subclone type in a matter of 2 or 3 hours instead of 2 or 3 days by using PCR that identifies the bacteria at the same time with a PCR collection of the base pair differences identifying each of the clones and subclones of the bacteria collected from a genomic alignment assay.

Accordingly, in one embodiment of the invention there is a method for collecting unique identification markers identifying the clones and subclones of a single species of infectious microorganism bacteria comprising:
a) collecting genomic alignment information of the clone and subclones of the single bacterial species; and
b) determining the base pair differences which uniquely identify each clone and subclone.

In yet another embodiment of the invention there is a method of identifying an unknown infectious microorganism specie and which clone or subclone the infectious microorganism is from with a DNA containing sample from the microorganism using a collection of base pair differences uniquely identifying each of the clones and subclones of one or more individual infectious microorganism comprising:

a) collect DNA sample from the microorganism;

b) treat the sample to release DNA for PCR identification;

c) combine the treated sample with real time PCR primers for a first collection of the clones of at least one infectious microorganism and real time PCR primers for a second collection of subclones of at least one infectious microorganism in the first collection; and

d) perform sufficient rounds of PCR on the combined treated sample with PCR primers to identify the unknown microorganism clone and subclone at the same time.

### BRIEF DESCRIPTION OF THE DRAWINGS

FIG. 1 is a flow chart of the method of collecting unique identification markers and identifying an unknown infectious microorganism.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “about” and “essentially” mean ±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “unique identification markers” refers to markers present in the DNA of an infectious organism such as a bacteria or a virus. More specifically, they are the base pair differences (a single point, i.e. change in a DNA base pair which is also called a single mutating nucleotide polymorphism) that uniquely distinguish one clone or subclone from another of a particular species of infectious organisms. For example, in S. aureus the DNA gyrase A gene (gyrA), results in resistance to the antibiotic ciprofloxacin and other antibiotics of the quinolone family. While effective on some subclones, this mutation is a major reason why fluoroquinolones are generally not infections considered for the treatment of S. aureus infections. It can be a single base pair difference or enough base pair differences to identify the particular organism and distinguish it from other clones or subclones of a particular species of infectious organisms. Another example is the alpha-hemolysin (hla) gene of S. aureus. Hla is an extremely potent toxin expressed at much higher levels of CA-MRSA compared to HA-MRSA. This point mutation delineates subclones of the CC30 group into those that are CA-MRSA related versus those closer related to HA-MRSA. An inactive hla toxin (e.g. MRSA isolates) correlated with a less virulent strain but that can cause more antibiotic resistant infections.

As used herein the term “infectious microorganism” refers to microscopic organisms such as bacteria and virus particles which contain DNA and are responsible for infections in other living organisms such as a mammal, insect, plant, or the like. Examples of infectious microorganisms include, but are not limited to, Staphylococcus aureus, Klebsiella pneumoniae, Escherichia coli, Mycobacterium tuberculosis, Streptococcus species and the like. Viruses include, for example, hepatitis viruses, influenza and several of the pathogenic viruses that cause meningitis. An infectious microorganism can include clones of the microorganism and subclones thereof.

As an example the phylogeny of S. aureus details the problems of just identifying the species and not which clone or subclone is being identified. The infectious microorganism S. aureus has two prominent clones: the CC30 lineage which is predominant in hospitals (HA-MSRA) and the CC8 lineage which is the predominant CA-MSRA strain of S. aureus in the United States. The CC8 clone has at least 11 subclones including 18813, 19321, 18807, 18811, FPR 3757, 18805, 18810, 18809 and 18806. While the CC30 lineage also has a number of subclones, the Southwest Pacific 22033, The Phage types 80/81 clones 22030, 21295 and 22251 and the contemporary hospital isolates 21203, 21345, 21247, 3636 and EMRSA-16. The CA-MRSA strains are more virulent than the HA-MRSA strains and infection with CA-MRSA strains can cause death within 24 hours. The HA-MRSA strains are less virulent but are resistant to more antibiotics. It is clear that just an identification of a strain as S. aureus is not sufficient to treat an infected organism such as a human. Waiting 2 to 3 days for a more detailed determination is risking patient complications and even death.

As used herein the term “genomic alignment information” of a species, clone or subclone, is the base pair of informational differences between the clones and subclones on a DNA level. That is the comparison of the base pairs such that there is identification differences uniquely identified for each clone and subclone. The alignment tools utilized, for example, included the Basic Local Alignment Search Tool (BLAST) and the program MUMMER. These programs align large segments of DNA and entire genomes can be compared to identify differences that occur due to single point mutations, genetic inversions, and/or large deletions/insertions of genetic material. An important thing to consider is that the database of sequences to be compared can be constantly updated to identify new mutations to use in the algorithm as well as refine the combinations of mutations utilized to identify the different clones and subclones. For example, the current PubMed Genome and PubMed Nucleotide databases have numerous genome and plasmid sequences for bacteria and genome sequences for viruses and these were utilized to identify which mutations delineate clones and subclones from one another in a single species. The conditions utilized to identify the specific mutations that would be useful for identifying these subclones can be changed as well. One can make less or more specific conditions to refine the algorithm. For example, the current set of markers identified utilized what is called a buffer of 50 in the alignment. The buffer means that if a mutation has another mutation within 50 base pairs it will not be considered for the algorithm. This is important to understand because there are “hot spots” within the genomes where mutations are more likely to occur. These genetic hot spots can change at a faster rate than other segments of the genome so monitoring these areas needs to utilize an extra level of scrutiny. Being able to update the analysis with newly sequenced strains of bacteria and viruses as they become available is important given that strains of bacteria can expand rapidly, as in the previous example of the CC8 lineage which has expanded over the past for about 15 years, or over the past for about 70 years as in the case of the CC30 lineage. Additionally, strains of bacteria have expanded to new locations with unmet speed given the new forms of air travel and the amount of travel between countries compared to the past centuries. Once these differences are obtained then PCR (polymerase chain reaction) primers for real time PCR can be made using techniques well known in the art. Primers are then utilized in standardized rounds of real time PCR to generate enough copies of DNA match using the primers such that they can easily be identified from an unknown sample of a microorganism.

As used herein the term “unknown infectious microorganism” refers to a sample suspected of having an infectious microorganism therein to which identification of the clone and subclone of the infectious microorganism is desired. A sample can be taken from an organism suspected of being infected such as a mammal or human and thus tissue, fluid or blood samples or the like are collected. Likewise, it could be from a surface such as a substrate in a hospital which may have an infectious organism sitting on the substrate.

As used herein a “DNA containing sample” is a sample suspected of having the microorganism. It may be collected from a substrate or another organism in any standard manner known in the art. The sample must then be treated to release DNA sufficient for PCR identification, i.e. care taken to not destroy the microorganism DNA. It may require macerating tissues, lysing blood cells where the organism might be contained and one of skill in the art can prepare a sample for testing of microorganism DNA such that it could be used with PCR primers for PCR identification. The unknown sample is then combined with information from one or more species of infectious microorganisms. The information for each species is the collection of base pair differences of the clones and subclones of each species. Real time PCR primers are prepared in the standard manner and the primers for the one or more species combined with a treated sample. This can be done all as one sample or several samples of each, for example, with one species. The PCR however can all be run at the same time so that at the end the species can be identified down to the subclone level.

Upon running the PCR with the primers and the sample, in a matter of 1 to 3 hours, the unknown sample will be identified down to the subclone level assuming the species is included in the one or more species collection of PCR primers. This method, however, can contain any number of desired species to be tested for and by running all at once the results will be in hours rather than days.

Once the species clone/subclone (or more if multiple infectious microorganisms are present) the particular identified microorganism can be treated with the appropriate treatment method. For example, organisms can be treated with antibacterials or antivirals and substrates can be cleaned with the appropriate antimicrobial cleaner.

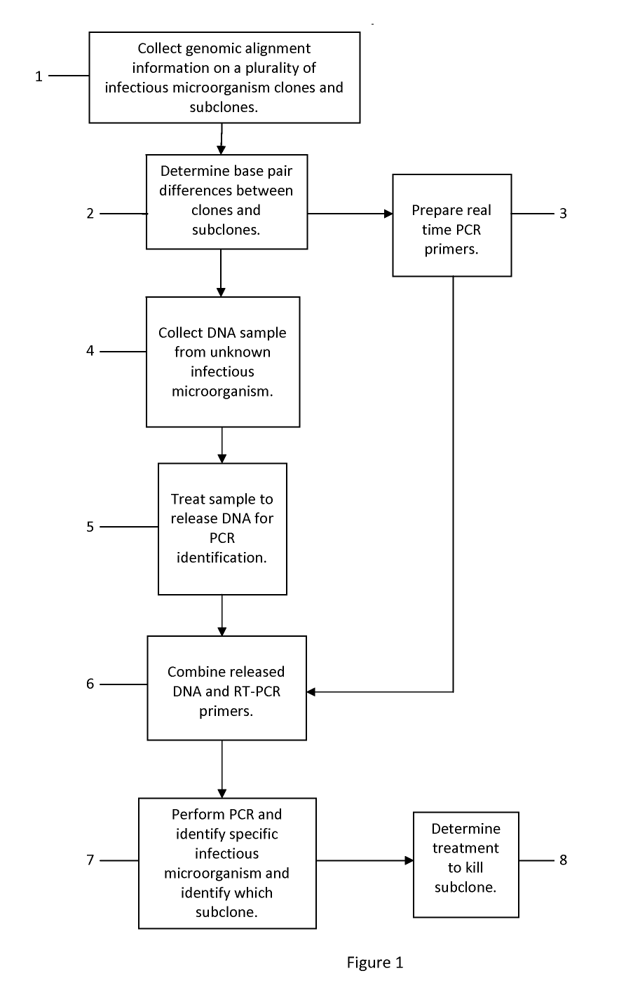

Now referring to the drawings, FIG. 1 is a combined flow chart of the method of collecting unique identification markers and identifying unknown infectious microorganisms. In the making of the collection, genomic alignment information is collected 1 on a plurality of infectious microorganisms clones and their subclones sufficient to identify each of them with as little a number of differences as possible, in most cases a single base pair difference, but sufficient to make the determination of the difference 2. The differences are then used to make real time primers (RT-primers) 3 for use in the general PCR process of identification.

Now in the process of identifying a sample for the presence of an infectious microorganism, a sample is taken from an organism such as blood or tissue or a surface is swabbed with the intent of collecting a DNA containing sample. It is generally assumed that the microorganism virus or bacteria will need to be treated to be available for PCR identification 5. So the cells can be lysed or otherwise treated to make DNA available. Such preparation is within the skill in the art.

Following the preparation, the released DNA is combined 6 with the RT-PCR primers 3 of the clones and sub clones. Clearly, the test is only as good as the number of clones in the test set of primers so multiple sets could be utilized based on location expectation of an infectious microorganism. Lastly, the combined DNA and primers is subject to the performance of PCR to identify the specific microorganism (clone) and then which subclone. The identification of the clone and then subclone can be done together or sequentially as desired and obviously doing them together is quicker than separate but separate means the separate step only needs RT-PCR primers to the particular subclones of the identified clone saving at least some on cost at the expense of time. Lastly, once the specific subclone has been identified a treatment (e.g. antibiotic) can be prescribed specifically for the subclone with confidence that the right one is being selected under the infectious circumstances.

### EXAMPLES

### Example 1

A subject presents at a clinic or hospital (or this could be a veterinary clinic as well given that many infectious diseases affect animals and can be transmitted to humans) with an abscess and a fever. It is determined that through the initial examination, the abscess needs to be lanced and that blood is drawn for diagnostic analysis. An aliquot of the lanced abscess is clarified by low speed centrifugation and then hemolyzed in order to lyses red and white blood cells. For the blood samples, a small aliquote of citrate or heparin anti-coagulated blood is hemolyzed to lyse the red and white blood cells. These two independent “clarified” samples have a series of RT-PCR primers added to them that will identify the specific subset of bacterial or viral pathogens. This first set of RT-PCR primers will examine ribosomal and genetic DNA elements that are specific for various genus and species of bacteria and these RT-PCR primers will identify if the bacteria is, for example, Staphylococcus aureus, Escherichia coli, Klebsiella pneumonia, Clostridium difficile or other pathogens. The next set of primers can be run at the same time or subsequent to this first set and these primers will determine which subclone of the infectious pathogen is causing the disease. For example, if the RT-PCR results show that the pathogen causing the infection is S. aureus, the second set of primers will tell which subclone of S. aureus, in this case a strain of the Southwest Pacific clone is causing the infection. Now, knowing which specific subclone is causing the infection, one can know what antibiotics to which this specific strain is most susceptible. As mentioned previously, the HA-MRSA and CA-MRSA strains are typically resistant to different antibiotics so knowing the specific clone causing the infection can save time by giving an indication of what kind of antibiotic to prescribe and not giving an antibiotic that would not produce positive results. This could also help with decreasing antibiotic persistence or development in the medical community. The overuse and bad conformity to doctors' orders regarding antibiotic prescriptions has led to a huge increase in amount and speed of antibiotic resistance in several different infectious diseases.

### Example 2

Another example would be in the area of infectious disease surveillance and epidemiology. Screening of patients as they enter hospitals could be accomplished by taking swabs from the anterior nares, skin, or throat cultures. The cultures grown from these diagnostic tests could be examined with the same PCR procedures described in the preceding paragraph to delineate if patients are colonized with these bacteria. These patients could be treated prior to surgery in order to further limit post-surgical complications that could result from infection. Likewise, surgical equipment and emergency room equipment could be screened in case they are contaminated with bodily fluids. As many infectious diseases are spread by contact, touching of one piece of equipment and then touching the patient could result in the spread of the infection. In the case that equipment is contaminated, the equipment can either be disposed of or decontaminated and future lessons can be learned to help limit future outbreaks. These aspects of surveillance could limit the spread of infection.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A method for collecting unique identification markers identifying the clones and subclones of a single species of infectious microorganism bacteria comprising
a) collecting genomic alignment information of the clone and subclones of the single bacterial species; and
b) determining the base pair differences which uniquely identify each clone and subclone.

2. The method according to claim 1 wherein the microorganism is a bacterium.

3. The method according to claim 2 wherein the bacterium is S aureus.

4. The method according to claim 1 which further comprises preparing real time PCR primers for each of the clones and subclones of the microorganism.

5. The collection of PCR primers made according to claim 4.

6. A method of identifying an unknown infectious microorganism species and which clone or subclone the infectious microorganism is from with a DNA containing sample from the microorganism using a collection of base pair differences uniquely identifying each of the clones and subclones of one or more individual infectious microorganisms comprising:
a) collect DNA sample from the microorganism;
b) treat the sample to release DNA for PCR identification;
c) combine the treated sample with real time PCR primers for a first collection of the clones of at least one infectious microorganism and real time PCR primers for a second collection of subclones of the at least one infectious microorganism in the first collection; and
d) perform sufficient rounds of PCR on the combined treated sample with PCR primers to identify the unknown microorganism clone and subclone at the same time.

7. The method according to claim 6 wherein the microorganism is a bacteria.

8. The method according to claim 7 wherein the bacteria is S. aureus.

9. The method according to claim 6 wherein the organism is a mammal.

10. The method according to claim 9 wherein the mammal is a human.

11. The method according to claim 6 wherein the sample is from an infected organism.

12. The method according to claim 11 wherein the infected organism is a mammal.

13. The method according to claim 12 wherein the infected organism is a human.

14. The method according to claim 11 wherein the microorganism is a bacteria which further comprising the treatment of the organism with an antibiotic to which the identified bacteria subclone is susceptible.

15. The method according to claim 6 wherein the sample is collected from an organism blood cell or tissue.

16. The method according to claim 5 wherein the sample is collected from a substrate suspected of harboring an infectious organism.
